(19) United States
(12) Patent Application Publication
(10) Pub. No.: US 2013/0041271 A1
(43) Pub. Date: Feb. 14, 2013

(54) DEVICES AND METHODS FOR MONITORING INTRACRANIAL PRESSURE AND ADDITIONAL INTRACRANIAL HEMODYNAMIC PARAMETERS

(71) Applicants: Shlomi Ben-Ari, Benyamina (IL); Shmuel Marcovitch, (US); Opher Kinrot, Ra'anana (IL)

(72) Inventors: Shlomi Ben-Ari, Benyamina (IL); Shmuel Marcovitch, (US); Opher Kinrot, Ra'anana (IL)

(21) Appl. No.: 13/445,171

(22) Filed: Apr. 12, 2012

Related U.S. Application Data
(60) Provisional application No. 61/474,739, filed on Apr. 12, 2011.
(60) Provisional application No. 61/540,090, filed on Sep. 28, 2011.

(51) Int. Cl. A61B 5/03; A61B 5/021; A61B 5/0402; A61B 5/0295

(52) U.S. Cl. 600/506

(57) ABSTRACT

Devices and methods for monitoring intracranial hemodynamic parameters, such as intracranial pressure, cerebral blood volume, cerebral blood flow, and cerebral perfusion pressure are disclosed. In one aspect, the devices and methods may involve receiving at least one impedance plethysmography signal. Waveforms may be extracted from the impedance plethysmography signals and used for estimating the intracranial hemodynamic parameters. Various characteristics may be determined from the waveforms to aid in the estimation of intracranial hemodynamic parameters.

### RELATED APPLICATIONS

This application claims the benefit of priority under 35 U.S.C. §119(e) of U.S. Provisional Application No. 61/474,739, filed Apr. 12, 2011, and U.S. Provisional Application No. 61/540,090, filed Sep. 28, 2011, both of'which are incorporated herein by reference in their entirety.

### TECHNICAL FIELD

Aspects of the present disclosure relate to detection, monitoring and/or analysis of signals characterizing cranial bioimpedance measurements, and the prediction of intracranial pressure and additional intracranial hemodynamic parameters based on such analysis.

### BACKGROUND

Many brain pathologies in the neuro-critical care units and ICU would benefit from non-invasive monitoring of Intracranial Pressure (ICP) and other intracranial hemodynamic parameters. Examples are traumatic brain injury (TBI), subarachnoid and intracerebral hemorrhage (SAH & ICH), ischemic strokes, brain tumors, and other conditions such as encephalitis, PRES and hydrocephalus. In addition, in other care settings such as ambulances, emergency room, and operating and recovery room patients would benefit from non-invasive intracranial hemodynamic monitoring in case of head trauma.

Cerebral pathologies can lead to temporary brain injury, permanent brain injury, or death. One symptom of these cerebral pathologies often includes increased intracranial pressure. When brain tissue is injured, for example, the injured tissue may develop edema and hemorrhage, both resulting in an increased ICP. To prevent additional brain damage one practice may include monitoring the ICP by insertion of a pressure probe into the brain. This is an invasive procedure typically involving drilling the skull (usually at an un-affected area), inserting the probe thru the drilled hole, and securing the probe with a nut to the skull. This invasive method typically involves risks associated with insertion of a probe into healthy brain tissue and risks of infection by an invasive probe.

A non-invasive method and apparatus may be used to measure and monitor ICP and additional intracranial hemodynamic parameters that may be clinically useful for diagnosing strokes, trauma, and other conditions that can affect the functioning of the brain. These parameters may include, for example, cerebral blood volume, cerebral blood flow, cerebral perfusion pressure, vascular autoregulation functioning and cerebral edema status.

One way to monitor or detect ICP and additional intracranial hemodynamic parameters may include physically inserting a probe into the cerebrospinal fluid or into an artery, angiography, computed tomography angiography (CTA), perfusion computed tomography (PCT), transcranial doppler ultrasound (TCD), positron emission tomography (PET), and magnetic resonance imaging (MRI) and angiography (MRA). Some non-invasive methods for detecting or monitoring ICP and additional intracranial hemodynamic parameters may require, for example, machines for carrying out CT, PCT, PET, and/or MRI procedures. In some instances, the lack of continuous monitoring, the cost of these machines, their limited mobility, and/or their significant expense per use, may limit their usefulness in situations where either regular, continuous, or frequent monitoring of intracranial hemodynamic characteristics may be desirable.

The foregoing description is merely exemplary for providing general background and is not restrictive of the various embodiments of systems, methods, devices, and features as described and claimed.

### SUMMARY OF A FEW ASPECTS OF THE DISCLOSURE

In the presently disclosed embodiments, several exemplary methods and systems are described that may be used to estimate ICP and additional intracranial hemodynamic parameters. In some embodiments, these methods and systems may be useful, for example, for continuous or frequent use and may involve, for example, electrodes, and/or a patient headset and cerebral perfusion monitor for acquiring impedance signals and extracting waveforms for estimating ICP and additional intracranial hemodynamic parameters. In addition, the patient headset and cerebral perfusion monitor may provide information for diagnosing changes in arterial occlusion, such as occlusions brought on by ischemic stroke or head trauma.

One exemplary disclosed embodiment may include an intracranial hemodynamic measurement apparatus. The apparatus may include at least one processor configured to receive at least one impedance plethysmography (IPG) signal associated with the brain of the subject. The at least one processor may be further configured to extract at least one waveform from the impedance plethysmography signal. The at least one waveform may be used, for example, to estimate at least one intracranial hemodynamic parameter.

In another embodiment, the at least one intracranial hemodynamic parameter may include intracranial pressure.

In other embodiments, the at least one processor may be further configured to determine at least one temporal characteristic of the extracted waveform, and to estimate the at least one intracranial hemodynamic parameter based on the at least one temporal characteristic of the extracted waveform. The at least one temporal characteristic may include at least one of a cardiac cycle length, a time interval between two peaks in the extracted waveform, and a time interval between a peak and a minimum in the extracted waveform.

In still other embodiments, the at least one processor may be further configured to determine at least one amplitude characteristic of the extracted waveform, and to estimate the at least one intracranial hemodynamic parameter based on the at least one amplitude characteristic of the extracted waveform. The at least one amplitude characteristic may include at least one of a mean value, a peak to peak range, a maximum value of a first derivative, a minimum value of a first derivative, a roughness measurement, and a kurtosis measurement.

In yet another embodiment, the at least one processor may be further configured to determine at least one amplitude characteristic and at least one temporal characteristic of the extracted waveform, determine a combined characteristic based on the at least one amplitude characteristic and the at least one temporal characteristic, and to estimate the at least one intracranial hemodynamic parameter based on the at least one combined characteristic of the extracted waveform. The at least one combined characteristic may include at least one of an exponentiated product of a time interval between a start of a cardiac cycle and a minimal value of a first derivative of the extracted waveform, an inverse of a cardiac cycle interval, and the minimal value of the first derivative and an exponentiated product of a time interval between a start of a cardiac cycle and a maximum value of a first derivative of the extracted waveform, an inverse cardiac cycle interval, and the maximum value of the first derivative.

In another embodiment, the at least one waveform may include a magnitude waveform, a phase waveform, a reactance waveform, or a resistance waveform.

In yet another embodiment, the at least one impedance plethysmography signal associated with the brain of the subject may include at least a left hemisphere impedance plethysmography signal and a right hemisphere impedance plethysmography signal.

In a further embodiment, the at least one processor may be further configured to receive at least one supplemental physiological signal associated with the subject, extract at least one supplemental waveform in the at least one supplemental physiological signal; estimate an intracranial hemodynamic parameter based on the at least one waveform and the at least one supplemental waveform. The at least one supplemental physiological signal may include an arterial blood pressure signal or an electrocardiogram signal.

In additional embodiments, the at least one processor may be further configured to determine at least one characteristic of the at least one waveform and the at least one supplemental waveform, and to estimate the at least one intracranial hemodynamic parameter based on the at least one characteristic.

Other embodiments involve alternative structures and methods described below. The foregoing summary and following description of drawings and following detailed description are exemplary of a just a few aspects of the disclosure, are explanatory only, and are not restrictive of the invention, as claimed.

### BRIEF DESCRIPTION OF THE DRAWINGS

The accompanying drawings, which are incorporated in and constitute a part of this specification, together with the description, serve to explain the principles of the embodiments described herein.

FIG. 1 provides a diagrammatic representation of an exemplary intracranial hemodynamic measurement apparatus consistent with exemplary embodiments of the invention.

FIG. 2 provides a diagrammatic representation of major cerebral arteries.

FIG. 3 provides a diagrammatic representation of exemplary bioimpedance signal pathways in the brain of a subject consistent with exemplary embodiments of the invention.

FIG. 4a provides a diagrammatic representation of an ICP waveform obtained from a healthy brain under normal conditions.

FIG. 4b provides a diagrammatic representation of an ICP waveform obtained from a pathological brain.

FIG. 4c provides a diagrammatic representation of an ICP waveform obtained from a brain under elevated ICP conditions.

FIG. 5a provides a diagrammatic representation of an exemplary ICP waveform.

FIG. 5b provides a diagrammatic representation of an exemplary impedance magnitude waveform, recorded simultaneously to the ICP waveform, consistent with embodiments of the invention.

FIG. 5c provides a diagrammatic representation of an exemplary impedance phase waveform, recorded simultaneously to the ICP waveform, consistent with embodiments of the invention.

FIG. 6 provides a diagrammatic representation of some exemplary amplitude characteristics that may be identified within a single cardiac cycle waveform of an impedance magnitude waveform or impedance phase waveform, consistent with embodiments of the invention.

FIG. 7 provides a diagrammatic representation of exemplary temporal characteristics that may be identified within extracted impedance magnitude waveform and impedance phase waveform, consistent with embodiments of the invention.

FIG. 8 provides a diagrammatic representation of an extracted impedance waveform cardiac cycle decomposed by a pulse decomposition algorithm, consistent with embodiments of the invention.

FIG. 9 illustrates a comparison between a measured ICP waveform and a supplemental arterial blood pressure waveform extracted from an arterial blood pressure signal, consistent with embodiments of the invention.

FIG. 10 provides a diagrammatic representation of exemplary features of a supplementary electrocardiogram signal, consistent with embodiments of the invention.

FIG. 11 provides a diagrammatic representation of the results of a generated IPG waveform analysis model in predicting measured ICP, consistent with embodiments of the invention

FIG. 12 is a flowchart showing the steps of an exemplary method for estimating an intracranial hemodynamic parameter, consistent with embodiments of the invention.

### DETAILED DESCRIPTION

Reference will now be made in detail to exemplary embodiments as with reference to the accompanying drawings. In some instances, the same reference numbers will be used throughout the drawings and the following description to refer to the same or like parts. These embodiments are described in sufficient detail to enable those skilled in the art to practice the invention and it is to be understood that other embodiments may be utilized and that changes may be made without departing from the scope of the present invention. The following detailed description, therefore, is not to be interpreted in a limiting sense.

Unless otherwise defined, all technical and/or scientific terms used herein have the same meaning as commonly understood by one of ordinary skill in the art to which the embodiments of the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Exemplary disclosed embodiments may include devices and methods for the reception and analysis of impedance plethysmography (IPG) signals representing bioimpedance. More specifically, they may include apparatuses for receiving and analyzing signals and outputting information for estimating physiological brain conditions.

Embodiments consistent with the present disclosure may include a measurement apparatus for non-invasive intracranial hemodynamic parameters. An intracranial hemodynamic measurement apparatus may include (but does not necessarily include), for example, support elements such as a headset, headband, or other framework elements to carry or house additional functional elements. Further structures that may be incorporated may include electrodes, circuitry, processors, sensors, wires, transmitters, receivers, and other devices suitable for obtaining, processing, transmitting, receiving, and analyzing electrical signals. An intracranial hemodynamic measurement apparatus may additionally include fasteners, adhesives, and other elements to facilitate attachment to a subject's body. As used herein, an intracranial hemodynamic measurement apparatus need not include all such features.

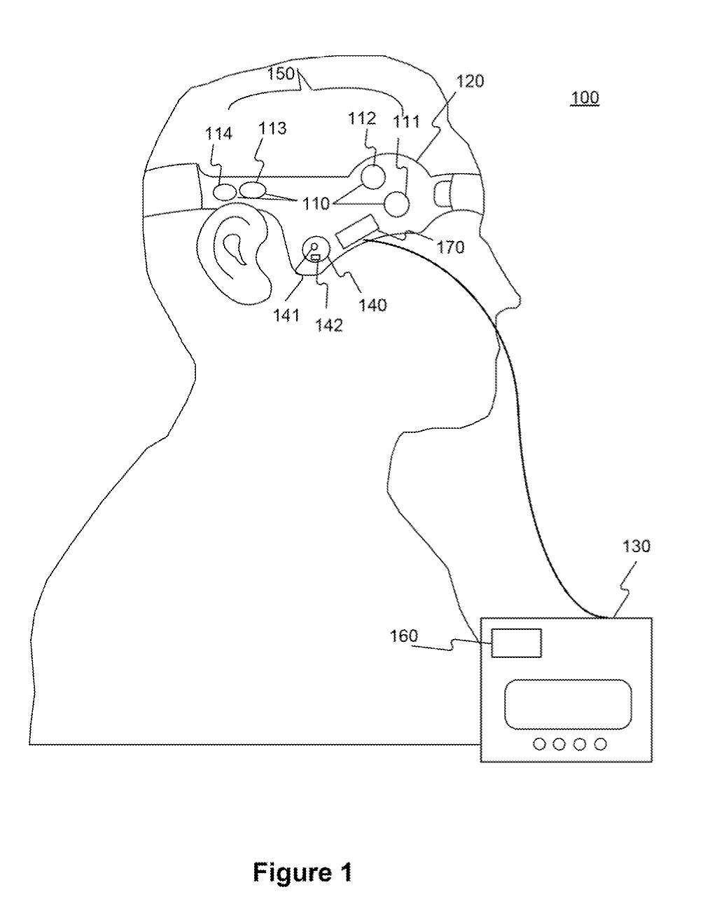

FIG. 1 provides a diagrammatic representation of an exemplary intracranial hemodynamic measurement apparatus 100. This exemplary apparatus 100 may include electrodes 110 affixed to a subject's head via a headset 120. Electrodes 110 may be connected to cerebral perfusion monitor 130 via wires (or may alternatively include a wireless connection).

In some exemplary embodiments consistent with the disclosure, an intracranial hemodynamic measurement apparatus may include at least one processor configured to perform an action. As used herein, the term “processor” may include an electric circuit that performs a logic operation on an input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The at least one processor may be configured to perform an action if it is provided with access to, is programmed with, includes, or is otherwise made capable carrying out instructions for performing the action. The at least one processor may be provided with such instructions either directly through information permanently or temporarily maintained in the processor, or through instructions accessed by or provided to the processor. Instructions provided to the processor may be provided in the form of a computer program comprising instructions tangibly embodied on an information carrier, e.g., in a machine-readable storage device, or any tangible computer-readable medium. A computer program may be written in any form of programming language, including compiled or interpreted languages, and it can be deployed in any form, including as a standalone program or as one or more modules, components, subroutines, or other unit suitable for use in a computing environment. The at least one processor may include specialized hardware, general hardware, or a combination of both to execute related instructions. The processor may also include an integrated communications interface, or a communications interface may be included separate and apart from the processor. The at least one processor may be configured to perform a specified function through a connection to a memory location or storage device in which instructions to perform that function are stored.

Consistent with some embodiments of the invention, the at least one processor be configured to receive a signal. As used herein, a signal may be any time-varying or spatially-varying quantity. Receiving a signal may include obtaining a signal through conductive means, such as wires or circuitry; reception of a wirelessly transmitted signal; and/or reception of a signal previously recorded, such as a signal stored in memory. Receiving a signal may further encompass other methods known in the art for signal reception.

At least one processor 160, schematically illustrated in FIG. 1, configured to receive and analyze one or more IPG signals associated with a brain of a subject, may be included in Cerebral Perfusion Monitor 130, as part of exemplary intracranial hemodynamic measurement apparatus 100. Processor 160 may be configured to perform all or some of the signal analysis methods described herein, or some of those functions may be performed by a separate processor. Processor 160 may also be configured to perform any common signal processing task known to those of skill in the art, such as filtering, noise-removal, etc. Processor 160 may further be configured to perform pre-processing tasks specific to the signal analysis techniques described herein. Such pre-processing tasks may include, but are not limited to, removal of signal artifacts, such as motion artifacts.

An IPG signal may represent bioimpedance information of a subject. When recorded from electrodes attached to the head of a subject, an IPG signal may be associated with the brain of the subject, and may represent bioimpedance information of the subject's brain tissue. An IPG signal may also contain information about the electrical impedance of the subject between any two portions of a subject's body, depending on the placement of suitable electrodes. Information about the electrical impedance of the subject may include information about the resistive and/or reactive components of electrical impedance. According to the present disclosure, in some exemplary embodiments an IPG signal may be measured as a response signal to at least one measurement voltage signal, and/or at least one measurement current signal. An IPG signal, as used herein, may include one or more of the response signal and the measurement signal. According to the present disclosure, an IPG signal may be obtained discontinuously or substantially constantly from a subject. Even when data is obtained continuously in an analog fashion it may be obtained at a fixed or variable digital sampling rate high enough to capture characteristics of interest within the signal. As used herein, a constantly obtained signal refers to a signal obtained substantially constantly. A constantly obtained signal may contain discontinuities, at either regular or irregular intervals, but contains enough data generate a temporal reconstruction of any characteristics of interest within the signal. For example, a constantly obtained IPG signal may be acquired using a digital sampling rate of 20 MSamples/sec (MS/sec) over a period of several minutes or hours. A sampling rate of 20 MS/sec may be sufficient to capture any voltage/current signals generated in the frequency range of 1 KHz-1 MHz. After obtaining the IPG signal by demodulating the voltage measurement with respect to the current measurement, it may be decimated to a lower sampling rate of, for example, 625 S/sec which is sufficient to capture any waveform characteristics that may be associated with a cardiac cycle of the subject, having time scales in hundredths of seconds. Characteristics of interest that may be captured in data extracted from a constantly obtained IPG signal will be discussed in further detail below.

According to the present disclosure, one or more waveforms may be extracted from an IPG signal. Extracted waveforms may include, for example, waveforms representative of impedance components and their change over time. Impedance components, may include, for example, the magnitude and phase of the impedance, or the resistive and reactive components of the impedance. Extracted waveforms may also be characterized by various combinations of these components. As used herein, a waveform may be considered “extracted” from an IPG signal if it may be derived from the IPG signal or if it may be determined using the IPG signal.

By way of example only, extracted waveforms representative of impedance components within an IPG signal may be expressed mathematically as follows. Extracted waveforms may be time-dependent, where I(t) describes a resistive component of the impedance, Q(t) represents a reactive component and represents the overall magnitude component of the impedance, where all three are measured in the units of Ohms. φ(t), the phase, is representative of a relationship between the resistive and reactive components of the signal I=real(ĒᵣQ=imag(Ē), where Z̅ is the impedance of the tissue. A different representation of the impedance may be given by

$$\vert \text{?} \vert = abs ( \text{?} ) , ϕ - {tan}^{- 1} ( \frac{Q}{t} ) . \\ \text{?} \text{indicates text missing or illegible when filed}$$

Waveforms may be also extracted at differing time scales, for instance to filter out either high or low frequency variations, or to focus on elements of the IPG signal having higher or lower amplitudes. Thus, the change in impedance components of a waveform may be examined on time scales on the order of fractions of seconds, seconds, minutes, and several hours. The change in impedance components of the waveform may also be examined on differing amplitude scales. For example, an impedance waveform associated with the cardiac cycle may show variation on relatively short time scales, on the order of fractions of seconds, and may show magnitude changes in an impedance amplitude waveform on the order of hundredths to tenths of ohm and thousandth to hundredths of a degree in an impedance phase waveform. In contrast, a baseline impedance waveform, associated with slow adjustments in cerebral blood volume, may demonstrate variations on longer time scales, such as on the order of several minutes or hours, and may be represented by a magnitude of tens to hundreds of ohms in an impedance amplitude waveform and 0-90 degrees in the impedance phase waveform.

In an impedance waveform extracted from an IPG signal, information about the subject's body may be contained in both amplitude and temporal characteristics of the impedance components of the waveform. Information about the subject's body may also be contained in a comparison between amplitude and temporal characteristics of the waveform, or in a comparison between characteristics of an impedance waveform with characteristics of a supplemental waveform, extracted, for instance, from another IPG signal, a blood pressure signal, an electrocardiogram signal, or a CO2 concentration signal.

Information about the subject's body contained in extracted impedance waveforms may be indicative, for example, of intracranial hemodynamic parameters within a subject's brain. Hemodynamic parameters may include, for example, intracranial pressure, cerebral blood volume, cerebral blood flow, cerebral perfusion pressure, and any other parameter that might be at least partially reflective of cerebral conditions.

An IPG signal associated with a subject's brain may be obtained from the left or right hemisphere of a subject's brain, and may also include a signal obtained from a global cranial measurement receiving information from both hemispheres at once. An IPG signal obtained from one hemisphere of a subject's brain may be indicative of hemodynamic characteristics in the hemisphere from which it is obtained, or hemodynamic characteristics from the opposing hemisphere.

Processor 160 may be configured to receive a signal from one or more electrodes 110, included in exemplary headset 120 of FIG. 1. Electrodes 110, may be arranged singly, in pairs, or in other appropriate groupings, depending on implementation. The electrodes on exemplary headset 120 may be arranged so as to obtain IPG signals. IPG signals may be measured by two sensor sections 150, disposed on the right and left sides of the head to correspond with the right and left hemispheres of the brain, for example. While only one sensor section 150 is shown in FIG. 1, an opposite side of the subject's head might include a similar electrode arrangement. Each sensor section 150 may include one pair of front electrodes, front current electrode 111 and front voltage electrode 112, and one pair of rear electrodes, rear current electrode 114, and rear voltage electrode 113. The distance between the pairs may be adjusted such that a particular aspect of an intracranial hemodynamic condition is satisfied. The electrode configuration depicted in FIG. 1 is only one example of a suitable electrode configuration. Additional embodiments may include more or few electrodes 110, additionally or alternatively arranged in different areas of exemplary headset 120. Other embodiments may include electrodes 110 configured on an alternatively shaped headset to reach different areas of the subject's head then the exemplary headset 120.

Pairs of electrodes 110 may include a current output electrode and a voltage input electrode. For instance, front current electrode 111 and front voltage electrode 112 may form an electrode pair. In one embodiment, an output current may be generated by cerebral perfusion monitor 130 and passed between front current electrode 111 and rear current electrode 114. The output current may include an alternating current (AC) signal of constant amplitude and stable frequency in the range of 1 KHz to 1 MHz. An input voltage induced on the head due to the output current may be measured between front voltage electrode 112 and rear voltage electrode 113. An input voltage may be measured at the same frequency as the output current. A comparison between the output current signal, e.g. a measurement signal, and the input voltage signal, e.g. a response signal, may be used to extract an impedance waveform of the subject. More specifically, a magnitude of the bioimpedance may be computed as a ratio of the input voltage signal amplitude to the output current amplitude signal, and a phase of the bioimpedance may be computed as the phase difference by which the output current signal leads the input voltage signal. Additional impedance components may be computed from the output current signal and the input voltage signal, or from the bioimpedance magnitude and phase, as required.

An IPG signal may also include output current at more than a single AC frequency. The output current may include a set of predefined frequencies and amplitudes, for example in the range of 1 KHz to 1 MHz, with detection of the measured voltage at all of the frequencies or a part of the frequency range.

Blood and fluid flow into and out of the head, and more specifically, the brain, may result in changes in the cranial bioimpedance characterized by the IPG signal measured by electrodes 110. Bioimpedance changes may correlate with blood content and blood pressure in the head and brain, as well as the contents and pressure of other fluids within the brain. The cardiac cycle, respiration cycle, and ICP slow-waves cycle affect the content and pressure of both blood and other fluids in the brain. In general, because blood and other fluids have a relatively low impedance when compared with tissue found in the head, higher blood or fluid content results in a lower impedance magnitude. Impedance chances associated with differing blood and fluid content and pressure within the brain may also cause variations in the frequency response of the brain impedance. Comparing bioimpedance measurements at different frequencies may provide additional information indicative of hemodynamic characteristics.

The exemplary headset 120 may include further devices or elements for augmenting bioimpedance measurements or for performing measurements in addition to bioimpedance measurements, such as an additional sensor or sensors 140. In one embodiment, additional sensor 140 may include, for example, a light emitting diode 141 and a photo detector 142 for performing Photo Plethysmography (PPG) measurements either in conjunction with or in alternative to bioimpedance signal measurements. The exemplary headset 120 may further include various circuitry 170 for signal processing or other applications and may include the capability to transmit data wirelessly to cerebral perfusion monitor 130 or to other locations. In an additional embodiment, cerebral perfusion monitor 130 may be integrated with headset 120. Although illustrated in the example of FIG. 1, additional sensor 140 and circuitry 170 may be omitted.

Exemplary headset 120 may include various means for connecting, encompassing, and affixing electrodes 110 to a patient's head. For example, headset 120 may include two or more separate sections that are connected to form a loop or a band that circumscribes the patient's head. Any of these aspects, including bands, fasteners, electrode holders, wiring, hook-and-loop connector strips, buckles, buttons, clasps, etc. may be adjustable in order to fit a patient's head. Portions of exemplary headset 120 may be substantially flexible and portions of the exemplary headset 120 may be substantially inflexible. For example, electrode-including portions of exemplary apparatus 120 may be substantially inflexible in order to, among other things, substantially fix electrodes 110 in specific anatomical positions on the patient's head. In addition to or in the alternative, other portions; such as bands or connectors holding the exemplary headset 120 to a patient's head, may be substantially flexible, elastic and/or form fitting.

Any portion of exemplary headset 120 may be specifically designed, shaped or crafted to fit a specific or particular portion of the patient's anatomy. For example, portions of exemplary headset 120 may be crafted to fit near, around or adjacent to the patient's ear. Portions of exemplary headset 120 may be specifically designed, shaped or crafted to fit the temples, forehead and/or to position electrodes 110 in specific anatomical or other positions. Portions of the exemplary headset 120 may be shaped such that electrodes 110 (or other included measurement devices) occur in specific positions for detecting characteristics of blood and fluid flow in the head or brain of the patient. Examples of such blood flow may occur in any of the blood vessels discussed herein, such as the arteries and vasculature providing blood to the head and/or brain, regardless of whether the vessels are in the brain or feed the brain.

Exemplary headset 120 may include features suitable for improving comfort of the patient and/or adherence to the patient. For example exemplary headset 120 may include holes in the device that allow ventilation for the patient's skin. Exemplary headset 120 may further include padding, cushions, stabilizers, fur, foam felt, or any other material for increasing patient comfort.

As mentioned previously, exemplary headset 120 may include one or more additional sensors 140 in addition to or as an alternative to electrical or electrode including devices for measuring bioimpedance. For example, additional sensor 140 may include one or more components configured to obtain PPG data from an area of the patient. Additional sensors 140 may comprise any other suitable devices, and are not limited to the single sensor illustrated in FIG. 1. Other examples of additional sensor 140 include devices for measuring local temperature (e.g., thermocouples, thermometers, etc.) and/or devices for performing other biomeasurements.

Exemplary headset 120 may include any suitable form of communicative mechanism or apparatus. For example, headset 120 may be configured to communicate or receive data, instructions, signals or other information wirelessly to another device, analytical apparatus and/or computer. Suitable wireless communication methods may include radiofrequency, microwave, and optical communication, and may include standard protocols such as Bluetooth, WiFi, etc. In addition to, or in alternative to these configurations, exemplary headset 120 may further include wires, connectors or other conduits configured to communicate or receive data, instructions, signals or other information to another device, analytical apparatus and/or computer. Exemplary headset 120 may further include any suitable type of connector or connective capability. Such suitable types of connectors or connective capabilities may include any standard computer connection (e.g., universal serial bus connection, firewire connection, Ethernet or any other connection that permits data transmission). Such suitable types of connectors or connective capabilities may further or alternatively include specialized ports or connectors configured for the exemplary apparatus 100 or configured for other devices and applications.

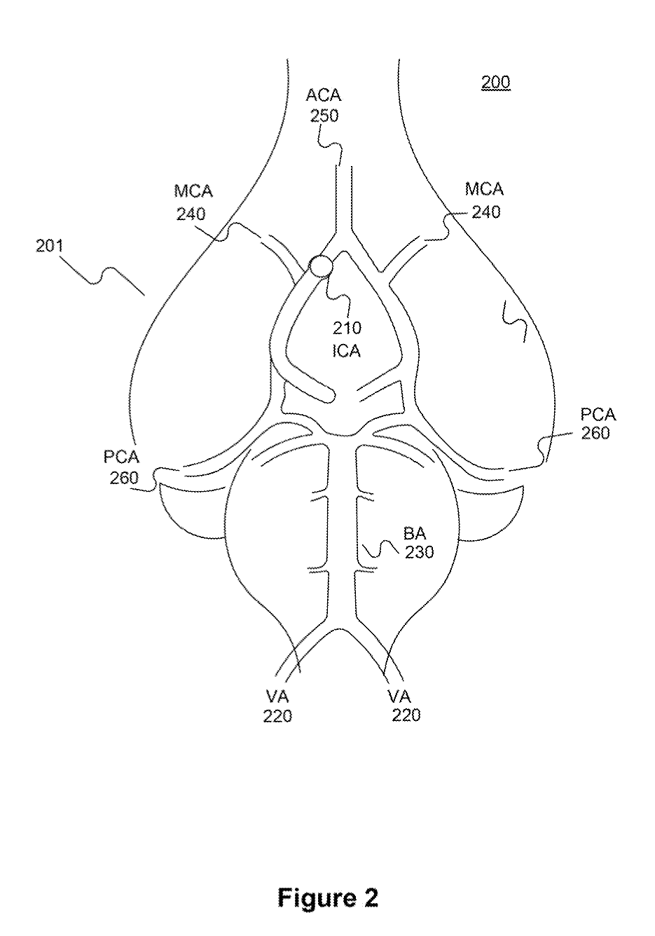

FIG. 2 provides a diagrammatic representation of major features of the cerebral vasculature 200. The cerebral vasculature in FIG. 2 is viewed from below the brain, with the top of the page representing the front of a subject. The blood supply to the brain 201 comes from four main arteries traversing the neck. The larger two are the right and left internal carotid arteries (ICA) 210, in the front part of the neck. The vertebral arteries (VA) 220 are located in the back of the neck and join to form the basilar artery (BA) 230. The internal carotid arteries and the basilar arteries are connected by Posterior Communicating Artery (not shown) and Anterior Communicating Artery (not shown) to form the Circle of Willis (COW). In an ideal patient, the COW is a network of connected arteries that allows blood supply to the brain 201 even when one or more of the feeding arteries is blocked.

The main arteries that supply blood to the brain 201 are the Middle Cerebral Arteries (MCAs) 240, Anterior Cerebral Arteries (ACAs) 250, and Posterior Cerebral Arteries (PCAs) 260.

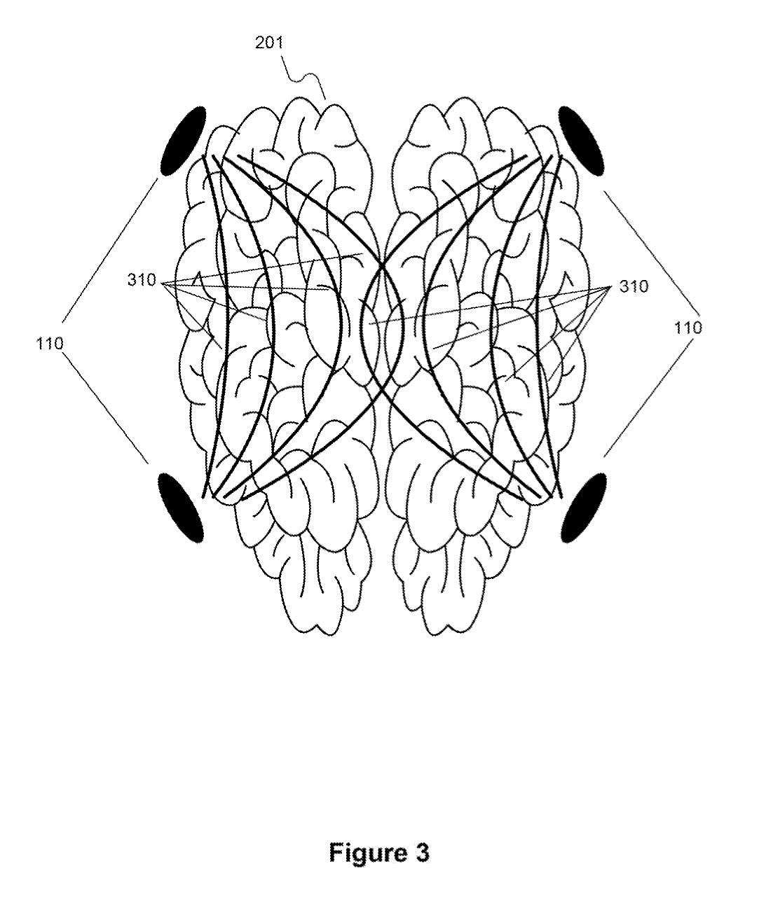

FIG. 3 provides a diagrammatic representation of exemplary impedance signal pathways 310 in the brain 201 of a subject. The exemplary configuration illustrates multiple signal pathways 310 through each of the right and left brain hemispheres. The multiple signal pathways extend between electrodes 110 affixed to the head of a subject via headset 120. The impedance of the signal pathways 310 may be influenced by the presence or absence of blood along the pathway, because blood has a relatively low impedance. At least some of the signal pathways 310 may be coincident with brain vasculature. Signal properties may thus be measured that are indicative of hemodynamic characteristics, such as pressure, blood flow, or volume, in the blood vessels of the brain 201. Changes in bioimpedance may thus be indicative of changes in pressure, blood flow, or blood volume, in the brain 201. Signal pathways 310 depicted in FIG. 3 are representative of only a small number of an infinite number of pathways which may exist in the general area of signal pathways 310.

In some embodiments consistent with the present disclosure, the at least one IPG signal associated with the brain of the subject may include at least a left hemisphere IPG signal and a right hemisphere IPG signal. A left or right hemisphere IPG signal, as used herein, may include an IPG signal reflective of impedance characteristics of the side of the brain with which it is associated. Left and right hemisphere IPG signals may be obtained from either side of the head, as impedance characteristics of the left hemisphere may be obtained from a location on the right side of a subject's head, and vice versa. An IPG signal relating to a particular side of a subject's brain may also be obtained from other locations, such as on the neck of subject, where, for example, carotid arteries are located.

An IPG signal may also be obtained through rearrangement of the voltage and current electrode pairs. For example, a frontal pair of voltage and current electrodes may be used to provide a frontal IPG signal and a rear pair of voltage and current electrodes may be used to provide an intracranial IPG signal. The left/right arrangement and frontal/intracranial arrangements may be electronically or mechanically switched using processor 160. To obtain more than one IPG measurement, for example by measuring simultaneously both right and left IPG signals, an alternating current frequency used in each of the measurements may be different, to differentiate between the sides. Using this technique, the voltage signal obtained from each side may be demodulated with respect to the corresponding current or with respect to the current delivered in the opposite side.

According to embodiments consistent with the present disclosure, the at least one intracranial hemodynamic parameter may include intracranial pressure. Intracranial pressure (ICP) is the pressure inside the skull, and therefore is also the pressure inside the brain tissue and the cerebrospinal fluid (CSF). The ICP may be influenced by several factors, including but not limited to, the cardiac cycle, the respiration cycle, and the ICP slow-wave cycle corresponding to the body's natural vascular autoregulation of cerebral blood flow. These three factors may affect the ICP at different time scales. The highest frequency variations in the ICP signal may be associated with the cardiac cycle and the arterial blood pressure changes induced by the heart's beating. At lower frequencies, the influence of the respiration cycle and corresponding changes to intrathoracic pressure may be detected in the ICP. At even lower frequencies, ICP slow-waves or plateau-waves with periods in the order of tens of seconds to several minutes correspond to the reactivity time scale of the vascular autoregulation mechanism. ICP slow-waves are pressure variations having a period of between approximately twenty seconds and several minutes. ICP slow-waves may be associated with physiological cerebral changes caused by the vascular autoregulation mechanism.

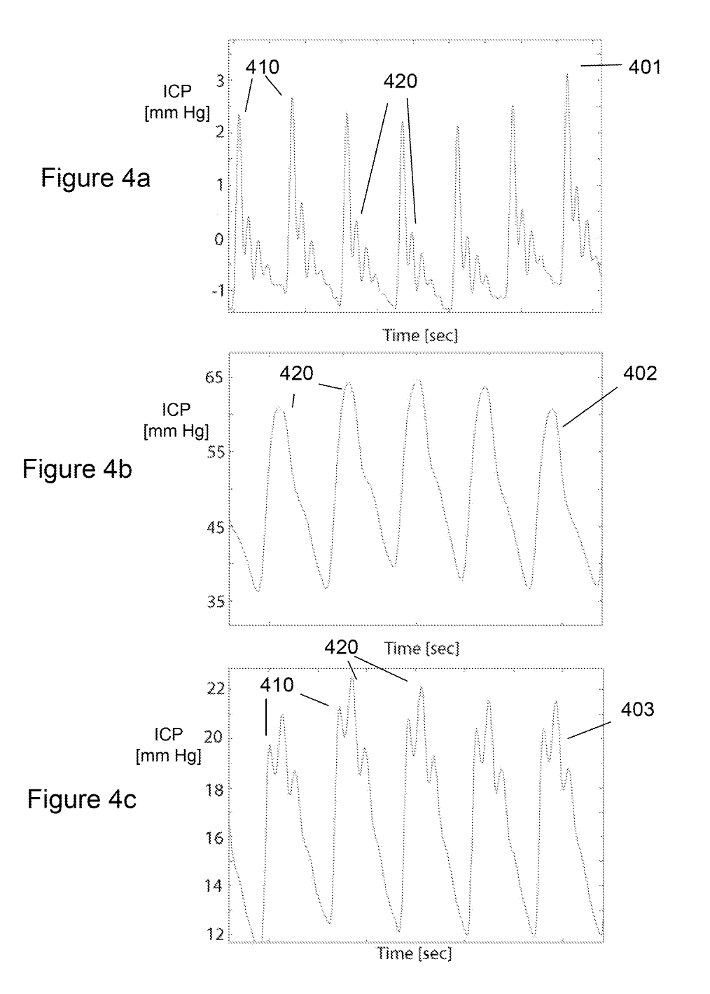

FIGS. 4a-4c illustrate ICP waveforms obtained through conventional, invasive measures. ICP waveform 401, illustrated in FIG. 4a provides a diagrammatic representation of an ICP waveform obtained from a healthy brain under normal conditions, with an ICP ranging between −1 and 2.5 mm Hg. ICP waveform 402, illustrated in FIG. 4b provides a diagrammatic representation of an ICP waveform obtained from a pathological brain, with an ICP ranging between 35 and 60 mm Hg. ICP waveform 403, illustrated in FIG. 4c provides a diagrammatic representation of an ICP waveform obtained from a brain under elevated ICP conditions, with the ICP ranging between 12 and 21 mm Hg.

Characteristics that are evident in these ICP waveforms vary depending on the condition of the subject's brain. For example, the ratio of a first peak (P1) 410 to a second peak (P2) 420 varies between the signals. In the healthy brain, P1 410 is significantly higher than P2 420. In the pathological brain, P2 420 is expanded in height and width to the point where it screens and obscures P1 410. Finally, in the elevated ICP brain, P1 410 is lower than P2 420. Thus, the ratio of P1 to P2 is an indicator that correlates with the mean value of the ICP. As another example evident in these waveforms, the roughness of each ICP waveform decreases with an increasing mean ICP. The roughness of a waveform measures the frequency of identifiable variations within the waveform. The P1 to P2 ratio and roughness of the ICP waveforms, as illustrated in FIGS. 4a-c, are exemplary identifiable characteristics in an ICP waveform, other such characteristics exist, as will be discussed further below.

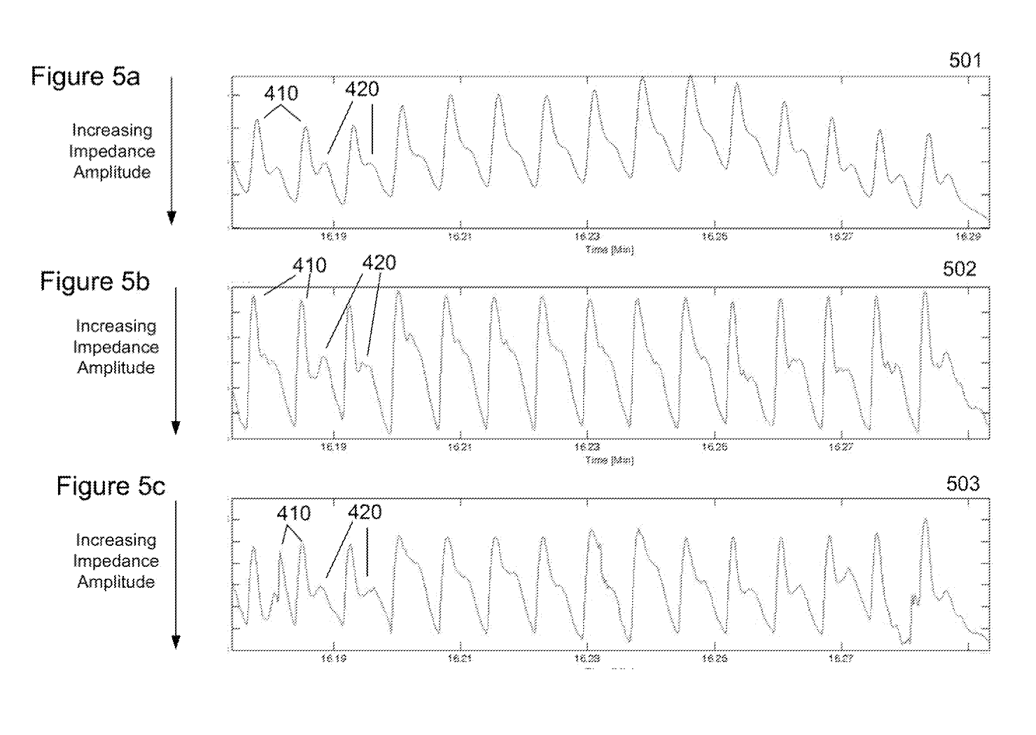

According to embodiments consistent with the present disclosure, at least one intracranial hemodynamic parameter may be estimated from at least one impedance waveform extracted from an IPG signal. FIGS. 5a-c illustrate an ICP signal recorded simultaneously with an IPG signal. FIG. 5a illustrates the ICP signal 501, while FIGS. 5b and 5c respectively illustrate an impedance magnitude waveform 502 extracted from the IPG signal and a phase waveform 503 extracted from the IPG signal. Each of these signals is illustrated over a time period corresponding with a single respiration cycle.

In FIGS. 5a-c, the impedance magnitude waveform 502 and the phase waveform 503 demonstrate characteristics that correlate with characteristics within the ICP signal 501. FIG. 5a provides a diagrammatic representation of an exemplary ICP signal 501. FIG. 5b provides a diagrammatic representation of an exemplary impedance magnitude waveform 502, recorded simultaneously to the ICP signal 501. FIG. 5c provides a diagrammatic representation of an exemplary impedance phase waveform 503, recorded simultaneously to the ICP signal 501.

For example, all three signals demonstrate P1 410 and P2 420 characteristics. A rise and fall of the mean ICP can also be seen in the ICP signal 501. Coinciding with the rise and fall of the mean ICP is a similar rise and fall in the height of P2 420 within that signal. Impedance magnitude waveform 502 and impedance phase waveform 503 also demonstrate a rise and fall in the height of P2 420 that coincides with the rise and fall of the mean ICP as shown in ICP signal waveform 501. Thus, information about the mean ICP may be obtained, for instance, from variations in the height of P2 420 within an impedance magnitude waveform 502 or an impedance phase waveform 503. These characteristics are detailed here for exemplary purposes only, as they are readily discernible from mere observation of waveforms 501, 502, and 503. Through additional analysis techniques, as will be discussed in more detail below, additional characteristics may be identified within impedance magnitude waveform 502 or impedance phase waveform 503.

According to some embodiments of the present disclosure, the at least one processor may be configured to determine at least one amplitude characteristic of an extracted impedance waveform. As used herein, an amplitude characteristic of a waveform is a quantity or value characterized by at least one measure of an amplitude of a waveform. For example, the amplitude of an identifiable feature, such as a peak, of a waveform may be an amplitude characteristic.

Amplitude characteristics may be determined in any waveform extracted from an IPG signal, including, for example, an impedance magnitude waveform, an impedance phase waveform, an impedance resistance waveform, and an impedance reactance waveform. Amplitude characteristics may be determined within a repeating cycle in an impedance waveform. For example, impedance magnitude waveform 502 displays a repetitious pattern of spikes. Each spike corresponds to an individual cardiac cycle of a subject and may be treated as a separate data set. Thus, identifying an amplitude characteristic within impedance magnitude waveform may include identifying the same characteristic, such as the height of a peak P1 410, in each spike corresponding to an individual cardiac cycle. Amplitude characteristics may also be determined in waveforms corresponding to the respiratory cycle or ICP slow-wave variations. ICP slow-wave variations may be associated with the body's autoregulation cycle. Amplitude characteristics may also be determined in by comparing features between multiple extracted waveforms. Furthermore, as will be described in more detail below, amplitude characteristics may be determined from supplemental waveforms, extracted, for example, from additional IPG signals, blood pressure signals, an ECG signal, or a CO2 concentration signal. For example, the peak to peak amplitude value of a blood pressure signal may be an amplitude characteristic. Determined amplitude characteristics may be used to estimate intracranial hemodynamic parameters.

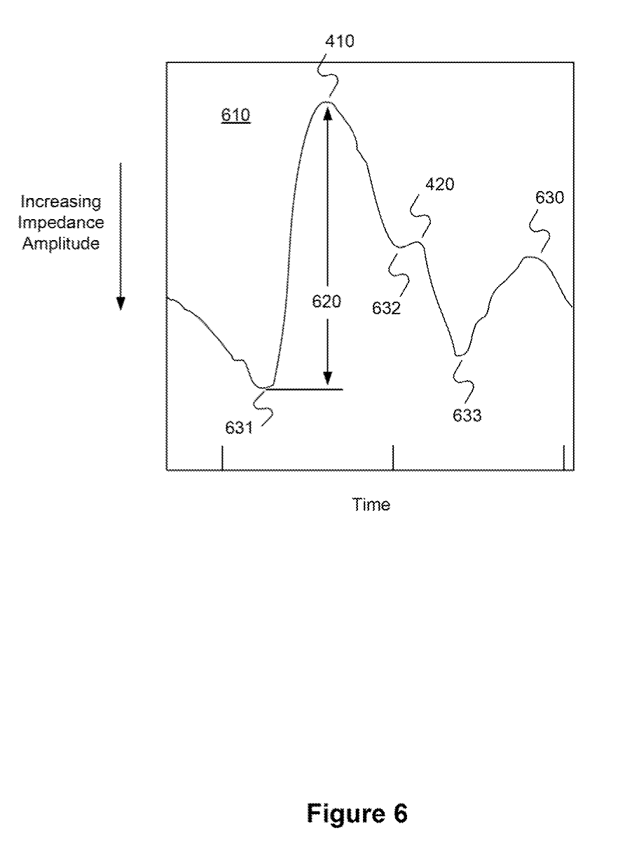

FIG. 6 provides a diagrammatic representation of some exemplary amplitude characteristics that may be identified within a single cardiac cycle waveform 610 of an impedance magnitude waveform 502 or impedance phase waveform 503. A peak to peak measure 620 may be measured between the maximum and the minimum magnitudes of impedance phase and amplitude in a time window. The maximal values of peaks of P1 410, P2 420, and P3 630 may constitute amplitude characteristics of an extracted waveform. The heights of local minima M0 631, M1 632, and M2 633 may constitute amplitude characteristics of an extracted waveform. Additional exemplary amplitude characteristics of extracted impedance waveforms may include ratios of any identified feature values, the maximum or minimum value of a first derivative of the waveform in a cardiac cycle, a standard deviation of the waveform in a cardiac cycle, respiratory cycle, or ICP slow-wave cycle, a kurtosis of the waveform in a cardiac cycle, respiratory cycle, or ICP slow-wave cycle, an area under of the waveform in a cardiac cycle, respiratory cycle, or ICP slow-wave cycle, a concavity measure of the waveform in a cardiac cycle, a roughness measure of the waveform in the cardiac cycle, and a peak to peak measurement in a respiratory cycle or ICP slow-wave cycle. Kurtosis is a statistical distribution measurement, providing information about the heaviness of a distribution tail. Concavity may be defined as the relation between the time period the waveform is above a certain threshold, e.g. a mean value or a midpoint value, and the length of a cardiac cycle.

Additionally, amplitude characteristics may be derived from amplitude measures of any other features identified in the present disclosure. The foregoing list is intended for exemplary purposes only, it will be understood by those of skill in the art that amplitude characteristics may be derived from any identifiable features within a single extracted waveform, and across multiple extracted waveforms.

According to some embodiments of the present disclosure, the at least one processor may be configured to determine at least one temporal characteristic of an extracted impedance waveform. As used herein, a temporal characteristic of a waveform is a quantity or value characterized by a timing relationship. For example, the elapsed time between two identifiable features, such as peaks, of a waveform may be a temporal characteristic. Temporal characteristics may be determined in any waveform extracted from an IPG signal, including, for example, an impedance magnitude waveform, an impedance phase waveform, an impedance resistance waveform, and an impedance reactivity waveform. Temporal characteristics may be determined within a repeating cycle within an impedance waveform. Identifying a temporal characteristic within impedance magnitude waveform 501 may include identifying the same characteristic, such as the time interval between peak P1 410 and Peak P2 420, in each spike corresponding to an individual cardiac cycle. Temporal characteristics may also be determined in waveforms corresponding to the respiratory cycle or ICP slow-wave variations. Temporal characteristics may also be determined in by comparing features between multiple extracted waveforms. Furthermore, as will be described in more detail below, temporal characteristics may be determined from supplemental waveforms, extracted, for example, from additional IPG signals, blood pressure signals, an ECG signal, and CO2 concentration signals. For example, the elapsed time between an R-wave peak of an ECG signal and an identifiable peak of an impedance magnitude waveform may be a temporal characteristic. Determined temporal characteristics may be used to estimate intracranial hemodynamic parameters.

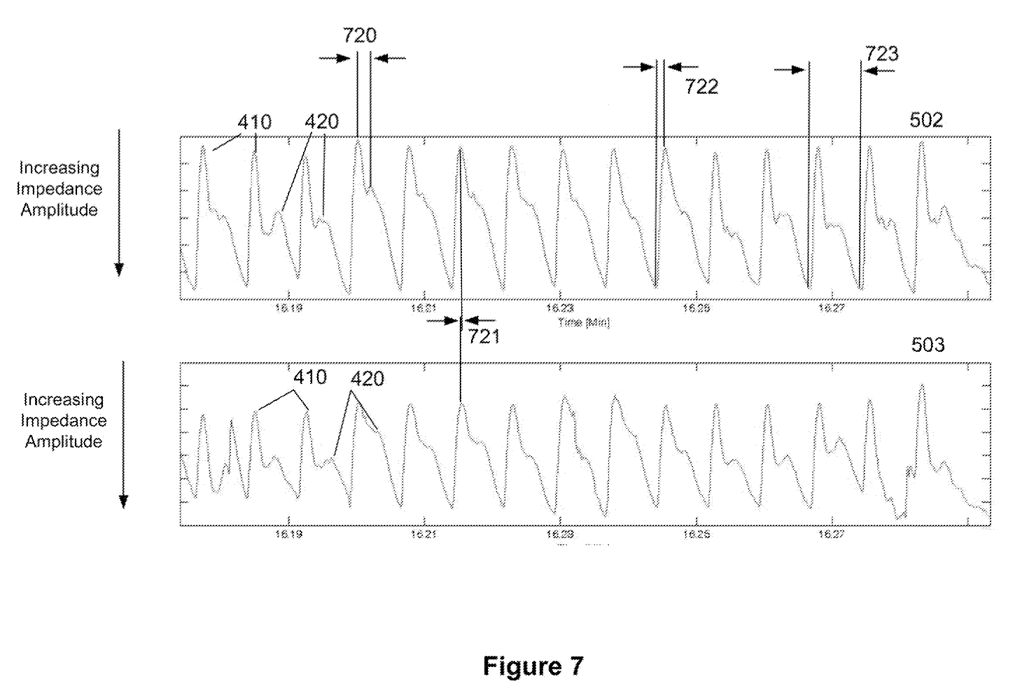

FIG. 7 provides a diagrammatic representation of exemplary temporal characteristics that may be identified within extracted impedance magnitude waveform 502 and impedance phase waveform 503. A P1-P2 time interval 720 may be measured between a P1 410 and a P2 420 within an extracted waveform. A P1-P1 time interval 721 may be measured between a P1 410 in impedance magnitude waveform 502 and a P1 410 in impedance phase waveform 503. A P1-M0 time interval 722 may be measured between P1 410 and M0 631 in an extracted waveform. Cardiac cycle length 723 may be measured between successive minimum values of an impedance waveform. Temporal characteristics may also be derived from time differences between any other features identified in the present disclosure. Additionally, temporal characteristics may be derived from any extracted waveforms, and are not limited to the impedance magnitude and phase waveforms discussed above. The foregoing list is intended for exemplary purposes only, it will be understood by those of skill in the art that temporal characteristics may be derived from a time difference between any identifiable features within a single extracted waveform, and across multiple extracted waveforms.

In some embodiments, a combined characteristic based on at least one amplitude characteristic and at least one temporal characteristic may be determined. A combined characteristic may be represented by any combination of temporal and amplitude characteristics, such as those previously described. A combined characteristic, for example, may include a time interval until the occurrence of a maximum or minimum value of a first derivative or a mathematical combination of a time interval until a first peak P1 occurs and the height of the first peak P1. Additionally, combined characteristics may include exponential characteristics, computed by exponentiating a constant or another characteristic with a product of a temporal characteristic and an amplitude characteristic. For example, the time interval between a start of a cardiac cycle and a maximum or minimum value of a derivative of an impedance waveform may be normalized by the cardiac cycle length and multiplied by the maximum or minimum value of the derivative. The resultant value may be used as the exponent of, for example, Euler's number e, to derive a combined characteristic. In this example a cardiac cycle length may be determined either from the impedance waveform itself, or from a supplemental ECG signal.

Amplitude and temporal characteristics, as described herein, may be determined through any suitable signal analysis technique. Signals may be filtered and smoothed prior to determining characteristics. Characteristics may be determined, for example, through functions that identify peak values, functions that isolate time intervals, functions that perform frequency or spectral analysis, and functions that perform experimental mode decomposition. Multi-variate analysis may be used to determine complex characteristics that account for multiple features of an impedance waveform simultaneously.

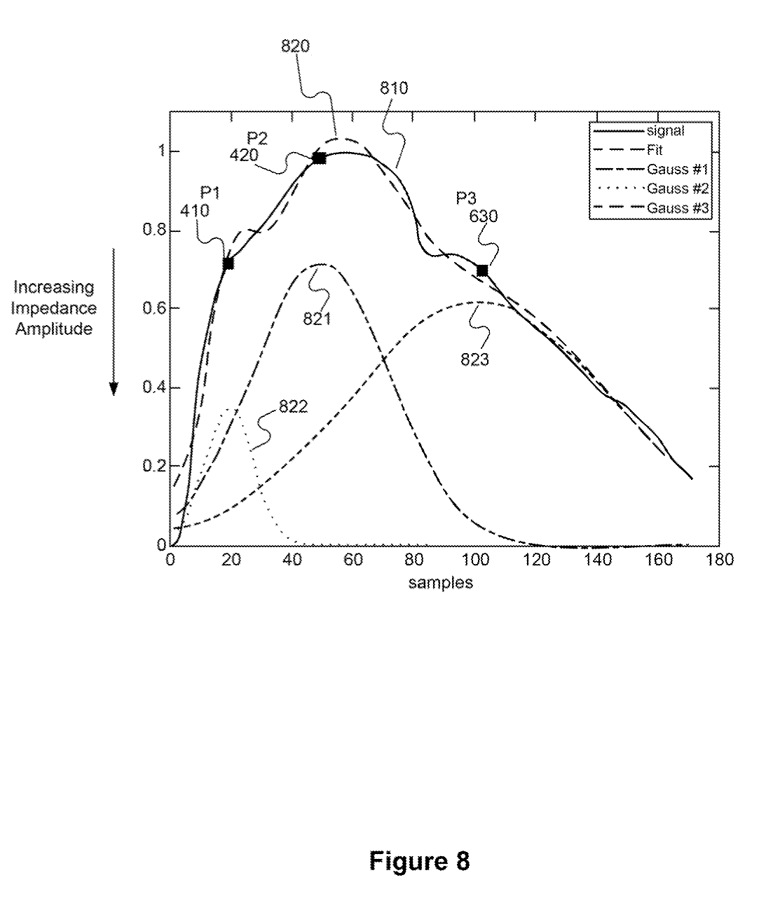

FIG. 8 provides a diagrammatic representation of an extracted impedance waveform cardiac cycle 810 decomposed by a pulse decomposition algorithm for detecting peaks P1 410, P2 420, and P3 630 to be used in determining temporal and amplitude characteristics. While these peaks may be determined by methods discussed above, a pulse decomposition algorithm represents an exemplary alternative method of identifying these peaks. A pulse decomposition algorithm may parameterize an impedance waveform by using a combination of basic functions to approximate the impedance waveform.

A base function used for a best fit may be related to physiological pulse waveform functions or may have a general shape that resembles a physiological pulse and provides stable fit parameters. One example of a suitable base function is a Gaussian function. A Gaussian base function may provide a clear definition of pulse width and curvature, a stable fit algorithm, and full determination of higher derivatives. A pulse decomposition algorithm utilizing Gaussian base functions may be performed as described below, with reference to FIG. 8.

FIG. 8 provides a diagrammatic representation of three Gaussian base functions, first Gaussian 821, second Gaussian 822, and third Gaussian 823 computed as best fits to the second, first and third peak, P2 420, P1 410, and P3 630, respectively. Using ECG signals, an impedance waveform may be divided into individual waveforms 810, each corresponding to a cardiac cycle. A waveform minimum at the beginning of an impedance waveform cardiac cycle may then be determined. Next, a waveform global maximum point following the minimum may be determined. It may then be determined whether the waveform global maximum point represents a first, second or third peak, P1 410, P2 420, or P3 630, based on a correspondence between the timing of the global maximum and previously obtained statistics. Next, a standard base function, such as a Gaussian, may be used to provide a best fit to the individual waveform near the determined global maximum, using timing and width limitations from previously obtained statistics. In FIG. 8, first Gaussian 821 is fitted to the highest peak P2 420. A best fit of the remaining two peaks, using second Gaussian 822 and third Gaussian 823 may then be determined using the same base function to the waveform remainder.

When combined, the Gaussian base functions form expected characteristic fit curve 820, which approximates the impedance waveform. The parameters that define the component base functions of expected characteristic fit curve 820, as derived from the exemplary pulse decomposition algorithm may serve to characterize each cardiac cycle in the extracted impedance waveforms.

The extracted impedance waveforms may then be replaced by a smooth waveform comprising the expected characteristic fit curves 820 of each cardiac cycle. This may permit the robust calculation of various features of interest such as minimum M0 631, minimum M1 632, minimum M2 633, and local curvatures at interest points. Methods such as the disclosed exemplary pulse decomposition algorithm may be useful for detecting features of an extracted impedance waveform that are difficult or impossible to detect through the use of other techniques, such as inflection point determination. As illustrated in FIG. 8, peaks P1 410, P2 420, and P3 630 do not coincide with local maxima of the extracted impedance waveform cardiac cycle 810, but with the peaks of the waveform's 810 component waveforms, Gaussians 821, 822 and 823.

Additional exemplary base functions may include a Generalized Extreme Value (GEV) distribution function. A GEV function may be used in conjunction with other base functions (such as Gaussians) or as the sole base function. For example, when decomposing a periodic extracted impedance waveform, Gaussian base functions may be used for fitting the first P1 410 and second P2 420 peaks in the early part of the waveform, and a GEV function for P3 630 in the later part. This choice may give a better fit for the diastolic part than using a Gaussian base function for P3 630, because GEV functions may be asymmetric while the Gaussian function is symmetric.

The parameterization of the extracted impedance waveform may also permit the collection and comparison of additional expected characteristics, including distribution statistics of the initial parameters. For example, the distribution of P2 420 pulse timing measured across multiple cardiac cycles may represent a temporal characteristic.

Other exemplary methods for determining features or characteristics of an extracted waveform may involve the use of other analysis techniques. For example, peaks and minimums may be identified through the use of first and second derivatives of a waveform, through numbering of maxima and minima of a waveform, and through any other suitable analysis technique.

In some embodiments consistent with the present disclosure, the at least one processor may receive at least one supplemental physiological signal and may determine at least one supplemental waveform in the supplemental physiological signal. Such a supplemental physiological signal may include, for example, an additional IPG signal, an arterial blood pressure signal, an ECG signal, and a CO2 concentration signal. Waveforms may be extracted from a supplemental physiological signal in the same manner as previously described with respect to an IPG signal.

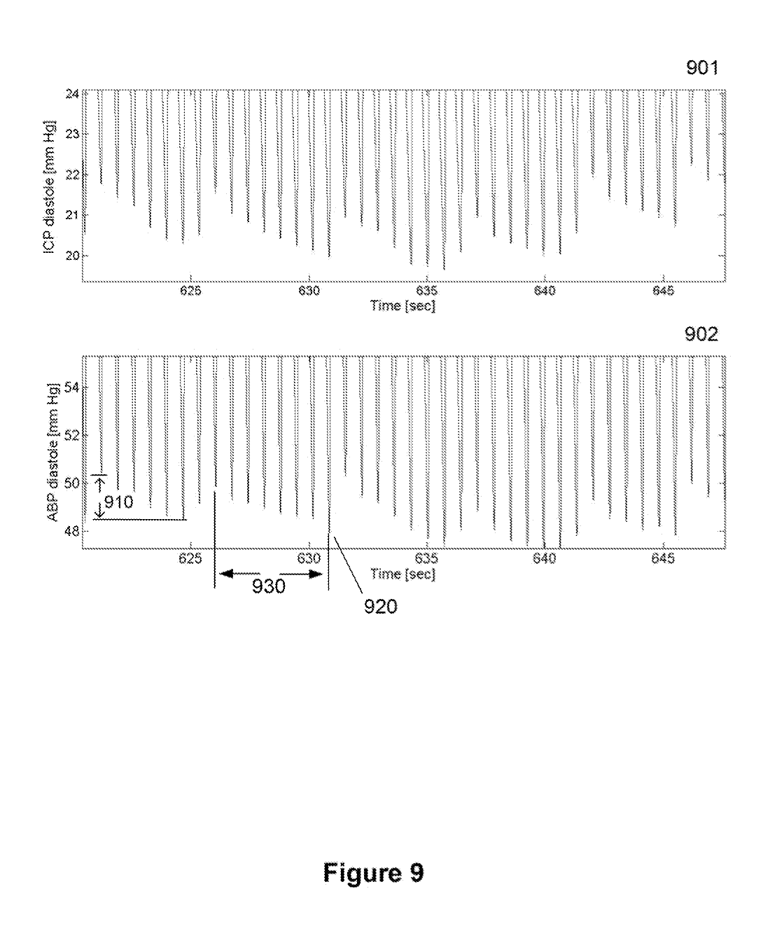

FIG. 9 illustrates a comparison between a measured ICP waveform 901 and a supplemental arterial blood pressure waveform 902 extracted from an arterial blood pressure signal. The comparison illustrates correspondences between the arterial blood pressure waveform 902 and the ICP waveform 901 over the course of several respiratory cycles. As illustrated, the minimum ICP value demonstrates a pattern similar to that of the minimum arterial blood pressure value. Because of correspondences between the arterial blood pressure and the ICP, temporal and amplitude characteristics helpful in estimating ICP may be determined from arterial blood pressure waveform 902.

Amplitude characteristics determined from an extracted arterial blood pressure waveform 902 may include a peak to peak measurement 910 in the respiration cycle and a diastolic pressure measurement 920 in the cardiac cycle, as illustrated in FIG. 9. Additional amplitude characteristics determined from an extracted arterial blood pressure waveform 902 may include a systolic blood pressure level, a mean blood pressure level, a peak to peak blood pressure range in a cardiac cycle or ICP slow-wave cycle, a standard deviation of arterial blood pressure within a cardiac cycle, respiratory cycle, or ICP slow-wave cycle, kurtosis of arterial blood pressure within a cardiac cycle, respiratory cycle, or ICP slow-wave cycle, an area under the arterial blood pressure waveform within a cardiac cycle, respiratory cycle, or ICP slow-wave cycle, and a maximum or minimum value of the first derivative of the arterial blood pressure within a cardiac cycle. Temporal characteristics determined from an extracted blood pressure waveform 902 may include a respiration complex duration 930.

Additionally, an arterial blood pressure waveform 902 may be used in combination with an impedance waveform to determine amplitude or temporal characteristics. For example, temporal characteristics determined from an impedance waveform and an arterial blood pressure waveform 902 may include a time interval between a maximum arterial blood pressure value and a maximum impedance waveform value, a time interval between a maximum of the arterial blood pressure waveform 902 and a maximum of an impedance waveform within a cardiac cycle, respiration cycle, or ICP slow-wave cycle, a time interval between a minimum of the arterial blood pressure waveform 902 and a minimum of an impedance waveform within a cardiac cycle, respiration cycle, or ICP slow-wave cycle, and a time interval between a respiration cycle or a ICP slow-wave cycle in an arterial blood pressure waveform and a respiration cycle in an impedance waveform.

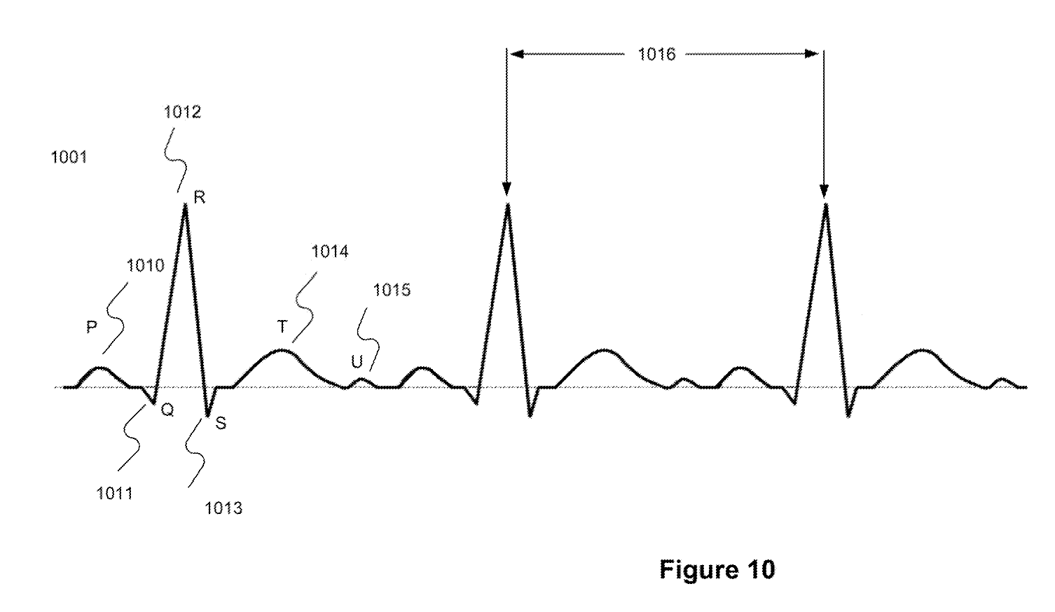

FIG. 10 provides a diagrammatic representation of exemplary features of a supplementary ECG signal 1001, which may provide additional information for estimating ICP. Illustrated in FIG. 10 are a P wave 1010, Q wave 1011, R wave 1012, S wave 1013, T wave 1014, and U wave 1015, as well as a cardiac cycle duration 1016. Any or all of these features may be used, for example, as reference points for determining temporal characteristics in conjunction with any of the extracted waveforms previously discussed. Temporal characteristics utilizing the cardiac cycle and an impedance waveform may include, for example, a time interval between the R-wave and a maximum value of an impedance waveform, a time interval between the R-wave and a maximum or minimum value of a first derivative of an impedance waveform, and a time interval between the R-wave and P1 410, P2 420, P3 630, M0 631, M1 631, M2 633 of an impedance waveform. Additionally, the cardiac cycle may be used to compute exponential temporal characteristics, by normalizing any of the previously described temporal characteristics with a cardiac cycle length and exponentiating a constant or another characteristic with the normalized value. For example, the time interval between the start of a cardiac cycle and a maximum or minimum value of a derivative of an impedance waveform may be divided by a cardiac cycle length, and the resultant value may be used as the exponent of, for example, Euler's number e, to derive a temporal characteristic.

Temporal characteristics utilizing the cardiac cycle and an arterial blood pressure waveform may include, for example, a time interval between the R-wave and a maximum value of an arterial blood pressure waveform, a time interval between the R-wave and a maximum or minimum value of a first derivative of an arterial blood pressure waveform, a time interval between the R-wave and a first, second, or third peak of an arterial blood pressure waveform, and a time interval between the R-wave and a first, second, or third local minimum of an arterial blood pressure waveform.

These lists of temporal and amplitude characteristics are intended to provide exemplary characteristics, and are not intended to be exhaustive or limiting. Temporal and amplitude characteristics may be determined from any combination of extracted waveforms, including impedance waveforms, blood pressure waveforms, ECG waveforms, and any other waveform extracted from a physiological signal. Temporal and amplitude characteristics may be determined using any suitable mathematical or signal analysis technique. A person of skill in the art will recognize that additional amplitude and temporal characteristics that may be determined from waveforms extracted from physiological signals.

As described above, in some embodiments consistent with the present disclosure, at least one intracranial hemodynamic parameter may be estimated based on at least one extracted impedance waveform. An IPG signal analysis model may be generated through a correlation between characteristics determined from extracted impedance waveforms and actual intracranial hemodynamic parameter measurements through, for example, regression and/or principle component analysis. Machine learning techniques may also be used to generate an IPG signal analysis model. This IPG signal analysis model may then be used to generate an estimate of a least one intracranial hemodynamic parameter based on the IPG waveform parameters.

An IPG signal analysis model for determining ICP, for example, may be generated and utilized as follows. As described above, amplitude and temporal characteristics may be determined from extracted impedance waveforms. These characteristics may be determined from a single cardiac cycle, a respiratory cycle, a ICP slow-wave/plateau-wave cycle, or any other time period of an extracted impedance waveform. These characteristics may then be used to build an IPG signal analysis model to determine a mean value of ICP over a time period, for example, a single cardiac cycle. After an IPG signal analysis model is generated based on correlations between determined characteristics and measured ICP, the model may be used to non-invasively estimate ICP based on determined characteristics. In this way, an IPG signal analysis model may be used on a continuous basis to provide an ICP estimate corresponding to each cardiac cycle shortly after it occurs.

An IPG signal analysis model may utilize any technique to develop a correlation between determined characteristics and an ICP waveform. These may include, for example, a linear combination of any or all of the characteristics described or contemplated herein, a linear combination of the results of a multiplication, division, or any other mathematical function involving any or all of the characteristics described or contemplated herein, a product of any or all of the characteristics described or contemplated herein, wherein each characteristic may be non-linearly transformed. Non-linear transformations may include, but are not limited to, exponentiation, the taking of a logarithm, and the raising to a constant power.

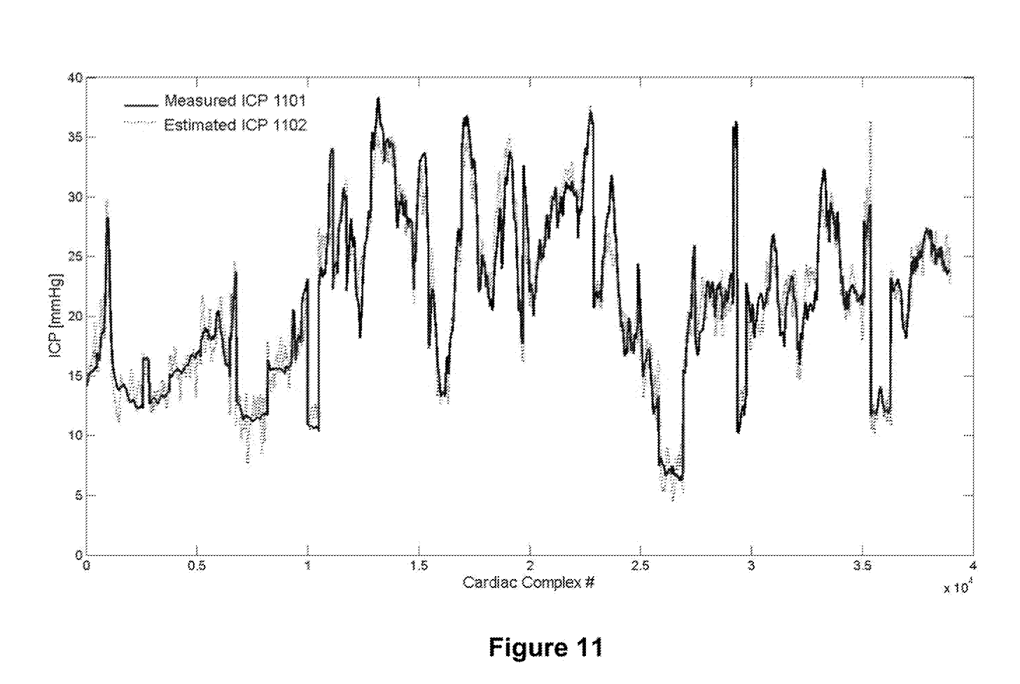

FIG. 11 provides a diagrammatic representation of the results of a generated IPG signal analysis model in predicting measured ICP. In FIG. 11, the solid black line represents a measured ICP waveform 1101, and the dotted black line represents an estimated ICP waveform 1102. ICP waveform 1101 was measured in a patient having brain trauma resulting in an unstable ICP. Estimated ICP waveform 1102 was determined from an IPG signal analysis model utilizing temporal and amplitude characteristics determined from impedance waveforms extracted from an IPG signal recorded simultaneously to measured ICP waveform 1102. The y-axis represents ICP in mm Hg, and the x-axis represents the cardiac cycle number for which an ICP value was measured or estimated. The x-axis shows a scale of 0-40,000 cardiac cycles. Several discontinuities in the graphs, for example at approximately 2,500, 10,000, and 30,000 cardiac cycles, are representative of discontinuities in the data, and do not correspond to physiological change. The strong agreement between measured ICP waveform 1101 and estimated ICP waveform 1102 demonstrates the success of the intracranial hemodynamic parameter estimation apparatus and methods described herein when applied to ICP estimation.

Waveform characteristics may be analyzed on a continuous basis to continuously provide an estimate of at least one intracranial hemodynamic parameter. For example, impedance waveform data may be continuously analyzed to estimate at least one intracranial hemodynamic parameter for every cardiac cycle within an uninterrupted time interval. The results from monitoring one portion of the uninterrupted time interval may be compared to the results from monitoring another portion of the uninterrupted time interval. For example, waveform characteristics may be continuously monitored throughout an uninterrupted time interval in order to monitor the intracranial pressure of a patient that has suffered traumatic brain injury.

Alternatively or additionally, waveform characteristics may also be monitored for intracranial hemodynamic parameter estimation over non-continuous time periods to provide diagnosis information. For example, waveforms extracted from IPG signals may be monitored during one time interval for comparison with waveforms extracted from IPG signals monitored during a second time interval that does not overlap or adjoin the first time interval. For example, an estimated intracranial hemodynamic parameter for a patient may be measured at a first time, e.g. prior to a surgery, upon admittance to a hospital, at a routine office visit, or at any other time when baseline measurement is possible. The estimated intracranial hemodynamic parameter may then be compared to parameters estimated at any later time, e.g. during a surgery, upon release from a hospital, at another routine office visit, etc.

The foregoing description provides some exemplary methods of receiving IPG signals, extracting waveforms, and estimating intracranial hemodynamic parameters. Alternate embodiments, however, may utilize other methods of performing these tasks. In some embodiments, alternate methods for determining waveform characteristics may be utilized. Thus, it will be appreciated by those of ordinary skill that various analysis techniques exist for analyzing a signal based on characteristics of extracted waveforms, and the invention in its broadest sense is not limited to any particular technique.

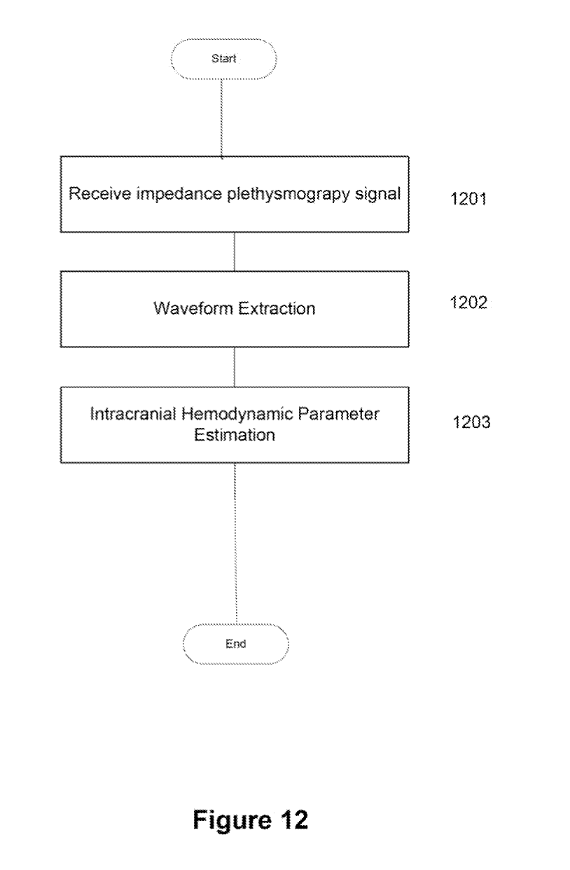

In an embodiment consistent with the present disclosure, a method for predicting a physiological brain condition is provided. FIG. 12 is a flowchart showing the steps of an exemplary method for estimating an intracranial hemodynamic parameter. At step 1201, at least one IPG signal is received. The at least one signal may be received, for instance, by a suitably configured processor 160. At step 1202, at least one waveform may be extracted from the at least one IPG signal. Processor 160 may be configured to perform this step.

At step 1203, at least one intracranial hemodynamic parameter may be estimated based on the at least one extracted waveform. The extracted waveform may be analyzed, for example, based on determined waveform characteristics, and may be analyzed by a suitably configured processor 160. Additional methods for estimating at least one intracranial hemodynamic parameter may include any and/or all of the techniques disclosed herein.

While this disclosure provides examples of the analysis of IPG signals, any signal that characterizes at least one cranial bioimpedance measurement may be assessed consistent with broad principles of this disclosure. While exemplary methods techniques in this disclosure are provided with respect to estimates of intracranial pressure, these methods and techniques may be used or adapted for estimation of any intracranial hemodynamic parameters. Further, the disclosure of uses of embodiments of the invention for detection, diagnosis, and monitoring of the discussed intracranial hemodynamic parameters is exemplary only. In its broadest sense, the invention may be used in connection with the detection, diagnosis, monitoring, and/or treatment of any physiological brain condition detectable using the principles described herein. Alternative embodiments will become apparent to those skilled in the art to which the present invention pertains without departing from its spirit and scope. Accordingly, the scope of the present invention is defined by the appended claims rather than the foregoing description.

What is claimed is:

1. An intracranial hemodynamic measurement apparatus, comprising:
at least one processor configured to:
receive at least one impedance plethysmography signal associated with a brain of a subject;
extract at least one waveform from the impedance plethysmography signal; and
estimate at least one intracranial hemodynamic parameter based on the at least one waveform.

2. The apparatus of claim 1, wherein the at least one intracranial hemodynamic parameter includes intracranial pressure.

3. The apparatus of claim 1, wherein the at least one processor configured to estimate the at least one intracranial hemodynamic parameter based on the at least one waveform is further configured to:
determine at least one temporal characteristic of the extracted waveform; and
estimate the at least one intracranial hemodynamic parameter based on the at least one temporal characteristic of the extracted waveform.

4. The apparatus of claim 3, wherein the at least one temporal characteristic includes at least one of a cardiac cycle length, a time interval between two peaks in the extracted waveform, and a time interval between a peak and a minimum in the extracted waveform.

5. The apparatus of claim 1, wherein the at least one processor configured to estimate the at least one intracranial hemodynamic parameter based on the at least one waveform is further configured to:
determine at least one amplitude characteristic of the extracted waveform; and
estimate the at least one intracranial hemodynamic parameter based on the at least one amplitude characteristic of the extracted waveform.

6. The apparatus of claim 5, wherein the at least one amplitude characteristic includes at least one of a mean value, a peak to peak range, a maximum value of a first derivative, a minimum value of a first derivative, a roughness measurement, and a kurtosis measurement.

7. The apparatus of claim 1, wherein the at least one processor configured to estimate the at least one intracranial hemodynamic parameter based on the at least one waveform is further configured to:
determine at least one amplitude characteristic and at least one temporal characteristic of the extracted waveform;
determine at least one combined characteristic based on at least one amplitude characteristic and the at least one temporal characteristic; and
estimate at least one intracranial hemodynamic parameter based on the at least one combined characteristic of the extracted waveform.

8. The apparatus of claim 7, wherein the at least one combined characteristic includes at least one of
an exponentiated product of a time interval between a start of a cardiac cycle and a minimal value of a first derivative of the extracted waveform, an inverse of a cardiac cycle interval, and the minimal value of the first derivative; and
an exponentiated product of the time interval between a start of a cardiac cycle and a maximum value of the first derivative of the extracted waveform, the inverse of the cardiac cycle interval, and the first derivative maximum value.

9. The apparatus of claim 1, wherein the at least one waveform includes at least one of a magnitude waveform, a phase angle waveform, a resistance waveform, and a reactance waveform.

10. The apparatus of claim 1, wherein the at least one impedance plethysmography signal associated with the brain of the subject includes at least a left hemisphere impedance plethysmography signal and a right hemisphere impedance plethysmography signal.

11. An intracranial hemodynamic measurement apparatus, comprising:
at least one processor configured to:
receive at least one impedance plethysmography signal associated with a brain of a subject;
receive at least one supplemental physiological signal associated with the subject;
estimate the at least one intracranial hemodynamic parameter based on the impedance plethysmography signal and the supplemental physiological signal.

12. The apparatus of claim 11, wherein the at least one supplemental physiological signal includes at least one of an arterial blood pressure signal and an ECG signal.

13. The apparatus of claim 11, wherein the at least one processor configured to estimate the at least one intracranial hemodynamic parameter based on the IPG signal and the supplemental physiological signal is further configured to:
extract at least one waveform from the impedance plethysmography signal;
extract at least one supplemental waveform from the supplemental physiological signal; and
estimate the at least one intracranial hemodynamic parameter based on the at least one waveform and the at least one supplemental waveform.

14. The apparatus of claim 13, wherein the at least one processor configured to estimate the at least one intracranial hemodynamic parameter based on the at least one waveform and the at least one supplemental waveform is further configured to:
determine at least one characteristic of the at least one waveform and the at least one supplemental waveform; and
estimate the at least one intracranial hemodynamic parameter based on the at least one characteristic of the at least one waveform and the at least one supplemental waveform.

15. A method of measuring intracranial hemodynamic parameters, comprising:
receiving at least one impedance plethysmography signal associated with a brain of a subject;
extracting at least one waveform from the impedance plethysmography signal; and
estimating at least one intracranial hemodynamic parameter based on the at least one waveform.

16. The method of claim 15, wherein the at least one intracranial hemodynamic parameter includes intracranial pressure.

17. The method of claim 15, further comprising
determining at least one temporal characteristic of the extracted waveform;
wherein estimating the at least one intracranial hemodynamic parameter includes estimating the at least one intracranial hemodynamic parameter based on the at least one temporal characteristic of the extracted waveform.

18. The method of claim 15, further comprising:
determining at least one amplitude characteristic of the extracted waveform;
wherein estimating the at least one intracranial hemodynamic parameter includes estimating the at least one intracranial hemodynamic parameter based on the at least one amplitude characteristic of the extracted waveform.

19. The method of claim 15, further comprising:
determining at least one amplitude characteristic and at least one temporal characteristic of the extracted waveform;
determining a combined characteristic of the at least one amplitude characteristic and the at least one temporal characteristic;
wherein estimating the at least one intracranial hemodynamic parameter includes estimating the at least one intracranial hemodynamic parameter based on the at least one combined characteristic of the extracted waveform.

20. The method of claim 15, further comprising:
receiving at least one supplemental physiological signal associated with the subject;
determining at least one supplemental waveform in the at least one supplemental physiological signal; and
estimating the at least one intracranial hemodynamic parameter based on the at least one waveform and the at least one supplemental waveform.
